(19) United States
(12) Patent Application Publication
(10) Pub. No.: US 2010/0286264 A1
(43) Pub. Date: Nov. 11, 2010

(54) COMPOSITION COMPRISING A NITROOXYDERIVATIVE OF ACETAMINOPHEN AND A ANTICONVULSANT DRUG FOR THE TREATMENT OF NEUROPATHIC PAIN

(75) Inventors: Francesco Impagnatiello, Milano (IT); Juan F. Herrero, Alcala de Henares (Madrid) (ES); Francesca Benedini, San Donato Milanese (Madrid) (ES)

Correspondence Address: ARENT FOX LLP, 1050 CONNECTICUT AVENUE, N.W., SUITE 400, WASHINGTON, DC, 20036, US

(73) Assignee: Nicox S.A., Sophia Antipolis - Valbonne (FR)

(21) Appl. No.: 12/811,358

(22) PCT Filed: Dec. 9, 2008
(86) PCT No.: PCT/EP2008/067060

§ 371 Date: Jun. 30, 2010

(30) Foreign Application Priority Data
Jan. 10, 2008 (EP) 08150156.1

(51) Int. Cl. A61K 31/222; A61P 25/06; A61P 25/08

(52) U.S. Cl. 514/509

(57) ABSTRACT

The present invention relates to compositions comprising a nitric oxide releasing paracetamol and an anticonvulsant drug. The compositions of the invention can be used use in the treatment of neuropathic pain in particular diabetic neuropathic pain, painful post-infarct syndrome, pain caused by chemotherapeutic treatment or pain arising from infections by viral agents.

The present invention relates to compositions comprising a nitric oxide-releasing paracetamol and an anticonvulsant drug selected from the group Gabapentin, Pregabalin and Tiagabine, the use of these compositions for the treatment of neuropathic pain.

Neuropathic pain is a form of chronic pain arising from a damage or injury to the peripheral or central nervous system. Neuropathic pain comprises a series of painful symptomatologies such as diabetic neuropathic pain, painful post-infarct syndrome, pain caused by chemotherapeutic treatment or pain is arising from infections by viral agents, for example Herpes zoster.

Neuropathic pain generally affects patients for many years and it is a social problem in that symptoms chronicity induces in subjects serious psychological stress, and it is characterized by a poor response to classic analgesics such as non-steroidal anti-inflammatory drugs and opiates.

In last years, several drugs for the treatment of neuropathic pain have been tested. Among these antidepressants and anticonvulsants are most commonly used.

Carbamazepine, the first anticonvulsant that has been widely used in clinical studies, has shown to be active in treating trigeminal neuralgia, painful diabetic neuropathic pain, and post-herpetic neuralgia. The administration of this drug has the drawback to present side effects such as somnolence, dizziness, ataxy, nausea and vomiting, thus limiting its use.

Gabapentin, Tiagabine and Pregabalin can be mentioned as newer anticonvulsant drugs used in the treatment of neuropathic pain. Gabapentin has the most clearly demonstrated analgesic effect for the treatment of neuropathic pain, specifically for treatment of painful diabetic neuropathy and postherpetic neuralgia. However, gabapentin is still not optimal as it is not very effective in numerous occasions and requires large dosages to provide significant efficacy in patients. Serious adverse effects, for example somnolence, weariness, obesity, have been observed following Gabapentin treatment. (Martindale XXXth Ed, page 374).

Despite the sophistication of new analgesic agents and to improved understanding of the neurobiological basis of pain, current pain management treatments have not been able to manage the side effect issues associated with the use of these agents.

There is currently a demand for additional drugs for the treatment of painful symptomatologies having a better is pharmacologic profile and reduced side effects.

Thus, it was an object of the present invention to find further drugs that are suitable for the treatment of neuropathic pain and which are more effective than the usual analgesic drugs actually employed in therapy and exhibit fewer undesired side effects.

It has been found that a composition comprising (a) a nitric oxide releasing paracetamol and (b) an anticonvulsant drug selected from Gabapentin, Pregabalin and Tiagabine is efficacious in the treatment of neuropathic pain, in particular diabetic neuropathic pain and painful post-infarct.

It has been found that the combination of a nitric oxide releasing derivatives of paracetamol with sub-effective doses of an anticonvulsant drug selected from the group of Gabapentin, Pregabalin and Tiagabine, results in a synergistic effect. Due to the synergistic effect the dose of the anticonvulsant drug are reduced and consequently the risk of undesired side effects are also reduced.

Accordingly, the present invention relates to a composition comprising:

(a) a nitric oxide releasing paracetamol and
(b) an anticonvulsant drug selected from Gabapentin, Pregabalin and Tiagabine.

The nitric oxide releasing paracetamol is selected from the group comprising the following compounds:

### 4-(nitrooxy)butanoic acid 4-(N-acetylamino)phenyl ester (1),

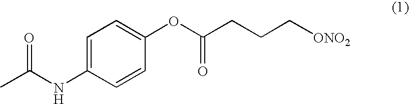

### 4-(nitrooxymethyl)-benzoic acid 4-(N-acetylamino)phenyl ester (2)

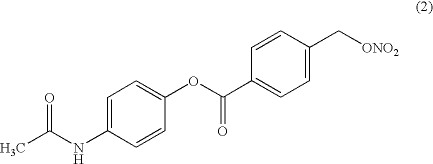

### 3-(nitrooxymethyl)-benzoic acid 4-(N-acetylamino)phenyl ester (3)

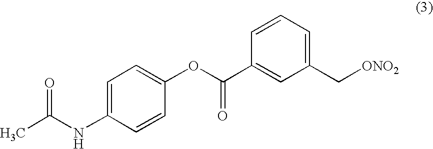

### 2-(nitrooxymethyl)-benzoic acid 4-(N-acetylamino)phenyl ester (4)

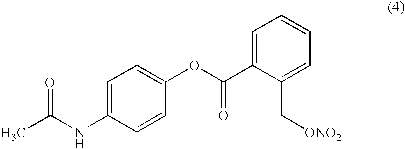

### trans-3-[4-(4′-nitrooxybutyryloxy)-3-methoxyphenyl]-2-propenoic acid 4-(N-acetylamino)phenyl ester (5)

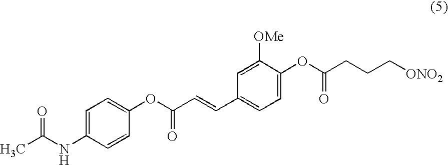

### 2-acetylamino-(4-nitrooxybutyryl)-3-mercaptopropionic acid 4-(N-acetylamino)phenyl ester (6)

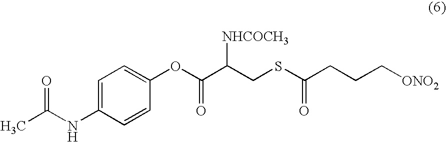

### 3-[(2-nitrooxy)ethyloxy]propanoic acid 4-(N-acetylamino)phenyl ester (7)

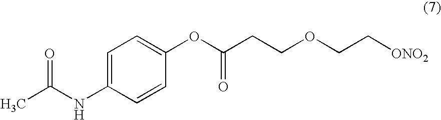

The composition of the present invention show a clearly better pharmacological profile than that hitherto obtained with the individual drugs when they are administered alone, and fewer adverse side effects.

A specific embodiment of the present invention is a composition comprising:

(a) 4-(nitrooxy)butanoic acid 4-(N-acetylamino)phenyl ester of formula (I) and

### (b) Gabapentin.

The nitric oxide releasing paracetamol derivatives as well as the methods for their preparation are disclosed in WO 02/30866.

### Paracetamol is Also Known as Acetaminophen.

In a further aspect the present invention relates to the use of the composition of the invention for the preparation of a medicament for the treatment of neuropathic pain that comprises the following painful symptomatologies: migraine pain, cancer pain, diabetic neuropathic pain, painful post-infarct syndrome, pain caused by chemotherapeutic treatment or pain arising from infections by viral agents.

In another embodiment the present invention relates to the use of the composition of the invention as described herein in the treatment of neuropathic pain, wherein the neuropathic pain comprises migraine pain, cancer pain, diabetic neuropathic pain, painful post-infarct syndrome, pain caused by chemotherapeutic treatment or pain arising from infections by viral agents.

Both components (a) and (b) as part of the composition may be administered in their usual daily dosage or preferably in sub-effective doses.

In the composition according to the invention the amount of nitrooxyderivative of acetaminophen of formula (I) is in the range from 10 to 1000 mg and the amount of Gabapentin, Pregabalin or Tiagabine, is in the range from 50 to 5000 mg.

The amount of the composition of the invention to be administered to the patient may vary depending on different factors well known to those skilled in the art, for example, the weight of the patient, the route of administration, or the severity of the illness.

According to the invention the nitric oxide releasing paracetamol and the anticonvulsant drug may be administered simultaneously or the nitric oxide releasing paracetamol and the anticonvulsant drug may be administered sequentially wherein the nitric oxide releasing paracetamol may be administered before or after the anticonvulsant drug in each case the two components may be administered by the same or different administration pathways.

Suitable pathways of administrations include but are not limited to oral, intravenous, intraperitoneal, transdermal, intrathekal, intramuscular, intranasal, transmucosal, subcutaneous, or rectal administration.

Thus, in a further aspect the present invention relates to a pharmaceutical composition comprising the composition of the invention as described herein and one or more pharmaceutical acceptable eccipients.

In one embodiment, the pharmaceutical dosage form is suitable for being administered orally, intravenously, intraperitoneally, transdermally, intrathekally, intramuscularly, intranasally, transmucosally, subcutaneously, or rectally.

### EXAMPLE F1

The antinociceptive effects of 4-(nitrooxy)butanoic acid 4-(N-acetylamino)phenyl ester (Compound 1), gabapentin and a combinations of compound 1 and gabapention were studied in spinal cord neuronal responses from adult male Wistar rats with mononeuropathy, using the recording of single motor units technique.

The application routes were intravenous (i.v.). The enhancement of the antinociception was studied by isobolographic analysis. Mononeuropathy was induced under anesthetic regime, seven days before the experiment, using the partial ligation of the sciatic nerve technique. The development of hyperalgesia was assessed by behavioral experiments, studying withdrawal reflex responses evoked by mechanical and thermal stimulation.

### Results:

4-(nitrooxy)butanoic acid 4-(N-acetylamino)phenyl ester (compound 1), dose-dependently reduced the nociceptive responses evoked by noxious mechanical and electrical stimulation, after intravenous (ID₅₀ of 542±5 μmol/kg for noxious mechanical stimulation). The combined administration of 4-(nitrooxy)butanoic acid 4-(N-acetylamino)phenyl ester and Gabapentin induced a more intense antinociceptive effect than any of the two drugs when given separately. The isobolographic analysis showed that the enhancement of the antinociception was synergic.

TABLE I
Nociceptive responses to noxious mechanical stimulation in rats
after administration of vehicle, compound 1, gabapentin and a composition
comprising the compound 1 and gabapentin. The nociceptive responses
are reported as percentage of vehicle values ± S.E.M.

| Gabapentin | Compoud 1 | | | | | | |
|---|---|---|---|---|---|---|---|
| (μmol/kg) | (μmol/kg) | 0 | 15 | 30 | 60 | 120 | 240 |
| 0 | | 100 ± 8 | 98 ± 6 | 95 ± 2 | 91 ± 4.5 | 86 ± 5 | 75 ± 9^{#} |
| 20 | 100 ± 5 | | 100 ± 1 | | | | |
| 40 | 95 ± 1 | | | 82 ± 5* | | | |
| 80 | 95 ± 2 | | | | 67 ± 11* | | |
| 160 | 79 ± 3 | | | | | 55 ± 6* | |
| 320 | 59 ± 6* | | | | | | 31 ± 5* |

*p < 0.05 vs single treatments with gabapentin or compound 1 at the respective dose.

1-12. (canceled)

13. A composition comprising
(a) a nitric oxide releasing paracetamol selected from the group comprising:
4-(nitrooxy)butanoic acid 4-(N-acetylamino)phenyl ester (1), 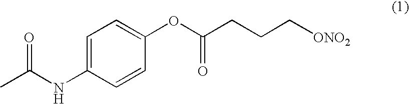
4-(nitrooxymethyl)-benzoic acid 4-(N-acetylamino)phenyl ester (2) 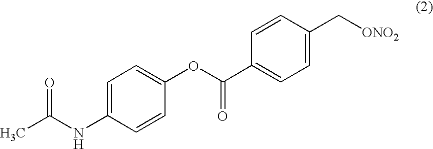
3-(nitrooxymethyl)-benzoic acid 4-(N-acetylamino)phenyl ester (3) 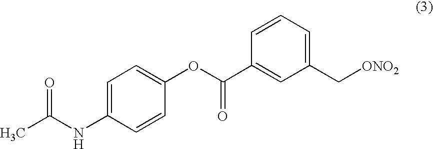
2-(nitrooxymethyl)-benzoic acid 4-(N-acetylamino)phenyl ester (4) 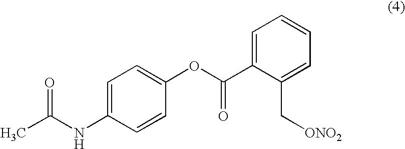
trans-3-[4-(4′-nitrooxybutyryloxy)-3-methoxyphenyl]-2-propenoic acid 4-(N-acetylamino)phenyl ester (5) 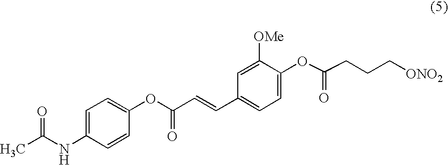
2-acetylamino-(4-nitrooxybutyryl)-3-mercaptopropionic acid 4-(N-acetylamino)phenyl ester (6) 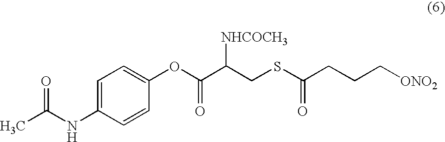
3-[(2-nitrooxy)ethyloxy]propanoic acid 4-(N-acetylamino)phenyl ester (7) 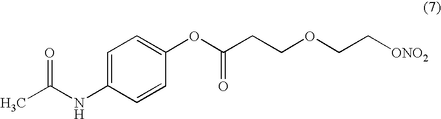
and
(b) the anticonvulsant drug Gabapentin.

14. Composition according to claim 13 for use as medicament.

15. Use of the composition according to claim 13 for the preparation of a medicament for treatment of neuropathic pain.

16. Use according to claim 15 wherein the neuropathic pain comprises migraine pain, cancer pain, diabetic neuropathic pain, painful post-infarct syndrome, pain caused by chemotherapeutic treatment or pain arising from infections by viral agents.

17. A pharmaceutical formulation comprising the composition according to claim 13 and pharmaceutical acceptable carrier.

18. The composition according to claim 13 wherein the nitric oxide releasing paracetamol and the anticonvulsant drug are administered simultaneously.
